(19) United States
(12) Patent Application Publication
(10) Pub. No.: US 2018/0169299 A1
(43) Pub. Date: Jun. 21, 2018

(54) METHOD FOR APPLYING CHARGED PARTICLES TO AN IMPLANT

(71) Applicant: Waidemar Link GmbH & Co. KG, Hamburg (DE)

(72) Inventor: Helmut D. LINK, Hamburg (DE)

(21) Appl. No.: 15/128,497

(22) PCT Filed: Mar. 25, 2015
(86) PCT No.: PCT/EP2015/056394

§ 371 Date: Sep. 23, 2016

(30) Foreign Application Priority Data
Mar. 25, 2014 (DE) 102014205533.425

(51) Int. Cl. A61L 27/28; A61L 27/54; A61L 27/12; A61L 27/32; A61L 27/10; A61L 27/16; A61L 27/56; C25D 13/04; C25D 13/12

(52) U.S. Cl. CPC A61L 27/28; A61L 2300/426; A61L 27/12; A61L 27/32; A61L 27/10; A61L 27/16; A61L 27/56; C25D 13/04; C25D 13/12; A61L 2420/08; A61L 2420/02; A61L 2300/606; A61L 2300/608; A61L 2300/406; A61L 2300/412; A61L 27/54

(57) ABSTRACT

The invention relates to a method for coating an implant (1), in particular a prosthesis, by applying charged particles (T) by means of iontophoresis to at least one section (10, 20; 110, 120) of the implant surface (3, 13). In the method, charged particles are accelerated in an electromagnetic field (E) and hit the surface (3, 13) of the implant in order to form a coating on the implant at least in some sections. The charged particles have at least one active ingredient.

### TECHNICAL FIELD

The invention relates to a method of manufacturing an implant, in particular a prosthesis, more particularly an endoprosthesis, by applying an electrically charged substance to at least one portion of a surface of the implant.

### PRIOR ART

Once an implant has been implanted, on the implant surface that is in contact with the surrounding tissue a race begins between pathogens possibly located on the surface and the integration of the implant into the tissue to anchor the implant.

To improve the chances of success of an implant, it may be beneficial to modify the implant surface or to apply a coating. Implant surfaces treated in this manner can be, e.g., conducive to anchoring the implant due to tissue growing in more easily, in particular bone tissue, or can prevent an infection caused by pathogens.

The latter can be achieved, for example, by embedding silver particles. Another possibility is to prevent inflammation by using active ingredients that result in the elimination of biofilm-forming bacteria. For this purpose antibiotics may be used, for example.

The bone tissue growing into the implant can be achieved, for example, by advantageous surface structuring and/or a corresponding implant material such as titanium. Here too, it is possible to use active ingredients to advantageously affect the anchoring of the implant by influencing bone remodeling by osteoblasts and osteoclasts.

A coating or introduction of silver particles on or into an implant surface can be achieved, for example, by galvanic or PVD coating. Other methods of surface treatment are, for example, sputter coating, electrostatic spray deposition or electrophoretic deposition.

However, both the active ingredients for preventing infections and osteoinductive active ingredients are temperature-sensitive. Thus, at least some of the aforementioned methods are disadvantageous in that the implementation thereof requires temperatures that decompose the active ingredients. Moreover, in particular with spray methods the problem arises that without additional effort they can coat complex three-dimensional surfaces or even simple undercuts in implants only irregularly or only on one side.

### SUMMARY OF THE INVENTION

Thus, the object of the invention is to provide a method that makes it possible to coat an implant using temperatures by which a reduced effect or decomposition of active ingredients is prevented.

Another object of the invention is to provide as regular a coating as possible with this method, using which implants having a complex, in particular porous, three-dimensional surface geometry can also be coated.

Furthermore, the applicable amount of a substance is to be increased by introduction into the depths of the substrate coating/surface structure so that the effect of the substance is expanded.

The aforementioned problems and objects were the incentive for the present invention. The idea underlying the invention is to introduce or apply a coating based on the principle of iontophoresis.

Iontophoresis is understood to mean the accelerated diffusion of charged particles due to an electric field which is generated, for example, by two opposite electrodes and the application of an accelerating voltage. The body to be treated by iontophoresis is then placed between the electrodes, with the charged particles to be applied having to be present between an electrode and the object to be coated, e.g. in an aqueous solution or a mist. The charged particles are accelerated in the electric field and thus arrive faster at the body or in the body than e.g. by diffusion alone.

Iontophoresis is used in particular as a medical method for active substance resorption through the skin by application of a weak direct electric current. The corresponding active substances must be present in ionized form, i.e. they must have an electric charge, or must be dissolved in a supporting electrolyte in which they are carried along. This is the only way in which the active substances can be accelerated in the electric field and thus traverse the skin in order to then get into the blood or into the tissue. The medicament is usually present in the form of an ointment. A typical application of iontophoresis is rheumatic diseases in which the active substance is supposed to enter a joint. Therefore, the particular advantage of iontophoresis is that an active substance can be specifically used locally instead of systemically.

For example, iontophoresis was also used in connection with a human allograft model and an allograft model from a sheep in order to introduce gentamicin and flucloxacillin into the model. It is attempted thereby to prevent the spread of pathogens that may adhere to the allograft and to thus promote its integration into the surrounding tissue.

The method according to the invention is defined by the independent claim 1 and the corresponding subclaims.

According thereto, a method is provided for coating an implant, in particular a prosthesis, by applying charged particles by means of iontophoresis to at least one portion of the implant surface. The charged particles are accelerated in an electromagnetic field and impinge on the surface of the implant so as to form a coating on at least part of the implant. Moreover, the charged particles comprise at least one active ingredient.

Prostheses, in particular endoprostheses, are artificial substitutes for damaged parts of the body, in contrast to allografts. Thus, in contrast to allografts, endoprostheses do not consist of human or animal tissue, but of biomaterials such as titanium, cobalt-chromium, ceramic or polyethylene, which are implanted and substitute diseased bone structures. Furthermore, with allografts it is not the aim to provide them with a comprehensive coating since the bone area of the allografts is supposed to be maintained, if possible, for a good integration with the surrounding tissue. In other words, they are characterized by fundamentally different requirements and properties regarding a coating.

Surprisingly, it has been shown that materials used for implants can also be coated by iontophoresis, during which the desired coating is applied by using charged particles. This “reverse iontophoresis” has the advantage that it gently applies an active ingredient onto an implant surface, thereby avoiding a negative impact on its efficacy.

Another advantage is that the particles introduced by means of reverse iontophoresis can again be released by means of iontophoresis, for example after the implant or the endoprosthesis has been implanted in a body. The original advantage of iontophoresis, i.e. the targeted use of an active ingredient, can thus be further realized.

It is possible that the charged particle is at the same time the active ingredient and/or that the active ingredient is connected to a charged particle.

In a preferred embodiment of the invention, the active ingredient comprises a medicament, in particular an antibiotic.

By using a medicament, the post-operative success after implantation can be positively affected in a targeted manner. For instance, infections can be prevented, which can be particularly beneficial to high-risk patients in whom an implantation may not otherwise be able to be performed due to a body healing disorder.

In a further preferred embodiment of the present invention, the active ingredient comprises a cytokine.

Using a cytokine, the healing reaction of the body, for example, can be positively influenced after an implantation. For instance, cytokines can be used to promote the ingrowth of surrounding bone tissue into the implant surface. For this purpose, so-called BMPs can be used, for example.

In a further particularly preferred embodiment, the implant surface is at least in part electrically conductive.

An electrically conductive implant surface preferably serves to generate the electric field so as to accelerate or move the charged particles. Thus, it can form one of the electrodes, or a part of an electrode, between which the electric field is generated. The electrically conductive surface therefore supports iontophoresis. One result of this may be, for example, that the coating is not performed at the electrically conductive portion of the implant surface, but preferably on other portions of the implant surface. The advantage of an implant surface that is in part electrically conductive is that the active ingredient can be specifically applied to or introduced into the surface. Thus, the method differs from electrophoresis.

If, furthermore, the region of the implant surface in which a coating is desired is also defined by conductivity, the reverse iontophoresis according to the invention can be combined with electrophoresis in an advantageous manner.

In a further preferred embodiment, at least one portion of the implant surface is formed of metal and/or polymer, in particular polyethylene, and/or ceramic.

Metal is advantageous in that it is electrically conductive and can thus be used as an electrode in iontophoresis. Moreover, it is possible to treat the surface of the metal in many ways known from the prior art to support good adhesion of the coating.

If the surface of the implant comprises a synthetic material or a polymer, this is advantageous in that also this surface can be coated with the active ingredient using the method according to the invention.

Ceramic is an implant material which may have, in particular as a bone substitute material, a porous surface so that charged particles can be deposited on the surface or can diffuse into the ceramic particularly well.

In a preferred embodiment, the accelerating voltage for accelerating the charged particles is at least 10 V and preferably maximally 500 V.

With such an accelerating voltage, a sufficient number of charged particles can be applied to the surface of the implant within a fixed period. Voltages that tend to be low are advantageous in that an unfavorable heat development that might affect the efficacy of the active ingredient can be largely avoided by these. It is further preferred to use an accelerating voltage of maximally 250 V and most preferably of maximally 100 V.

In a further embodiment, the accelerating voltage is applied for a period of 1 minute to 120 minutes.

Considering the accelerating voltage applied to avoid significant heat development, this application period has proven successful. In general, with a long application of the accelerating voltage, a shorter period is advantageous, and vice versa, to avoid excessive heat development that may negatively affect the active ingredient.

In a further particularly preferred embodiment of the present invention, the strength of the electromagnetic field is changed during application of the charged particles.

By changing the field strength, the coating process can be continued even with a longer coating duration and thus greater layer thickness. In other words, a possible decrease in the coating effect can be counteracted by a stronger electromagnetic field.

Furthermore, a more adhesive coating can be achieved by a stronger electromagnetic field. Thus, the durability of the coating can be influenced by selecting the field strength. This is of interest with regard to the release of the active ingredient as well as with regard to implants which are exposed to great mechanical forces during implantation, which may result in damage to the coating, e.g. at the shaft of a hip endoprosthesis.

In a further particularly preferred embodiment of the present invention, the surface of the implant is coated and/or modified for an improved intake of the charged particles preferably before iontophoresis is carried out.

This has the advantage that the implant surface can be optimally prepared for iontophoresis. Since preferably no active ingredient is involved in this step yet, it is possible to modify the implant surface with process parameters that could otherwise negatively affect the active ingredient. These include, for example, a mechanical modification of the implant surface and high temperatures occurring with classic coating methods such as plasma spraying. Using a method carried out in this manner, a kind of base coat can thus be applied to the implant surface, which allows for better adhesion of the coating applied by iontophoresis.

In a further preferred embodiment of the present invention, the implant is formed with a rough and/or porous surface.

Using a rough and/or porous surface in combination with the reverse iontophoresis according to the invention has the advantage that a surface with the active ingredient coating provided by iontophoresis, which is particularly advantageous for the ingrowth of bone tissue, allows for faster and more reliable anchoring of the implant in a bone.

It is also conceivable to configure the implant with a first layer and a second layer disposed thereabove, the first layer of which has a low porosity for taking up and releasing an antibiotic and the second layer disposed thereabove has a higher porosity compared to the first layer for binding osteoprogenitor cells.

### BRIEF DESCRIPTION OF THE DRAWINGS

FIG. 1 shows a schematic drawing of an apparatus for carrying out the method according to the invention on a first implant surface.

FIG. 2 shows a schematic drawing of an apparatus for carrying out the method according to the invention on a second implant surface.

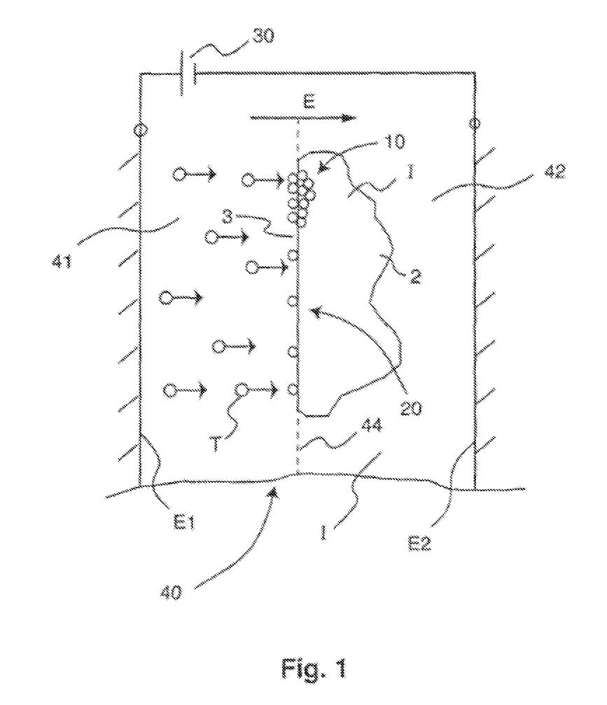

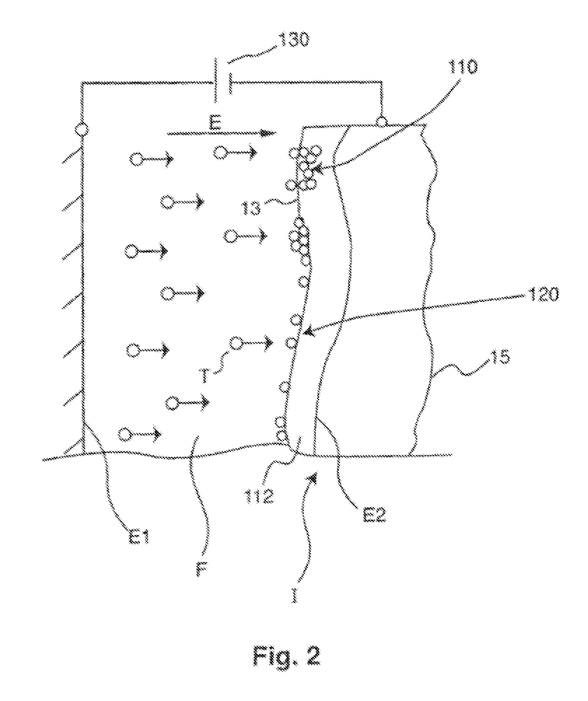

In FIGS. 1 and 2, identical features or features having the same effect are provided with identical or similar reference numerals.

### DETAILED DESCRIPTION OF PREFERRED EMBODIMENTS

In the following, preferred embodiments of the present invention will be described with reference to the corresponding drawings.

Iontophoresis is usually used to release an active ingredient specifically where it is supposed to unfold its effect. In contrast to this, a “reverse iontophoresis” is carried out in the method according to the invention, during which charged particles with an active ingredient are applied to or introduced into an implant.

Within the meaning of the present invention, charged particles are to be understood as being particles having a charge which allows the substance to be accelerated and moved using an electromagnetic field. For example, an ion is such a particle.

Applying the charged particles to the implant surface can also be referred to as introducing if the charged particles penetrate into a porous implant surface. This is also coating within the meaning of the present invention since the porous surface of the implant is coated on and below the implant surface.

FIG. 1 shows a first embodiment of the method according to the invention. In this method, two electrodes E1 and E2 having opposite polarities are disposed opposite each other. A coating space 40 is located between the electrodes, which contains, apart from the electrically charged particles T, preferably a fluid F. Alternatively, it is also possible to use a mist instead of a fluid, for example a spray mist, which contains the charged particles T or which consists of these charged particles.

The use of a mist has the advantage that it can be applied to the implant surface 3 easily and specifically in a predetermined pattern. The liquid, on the other hand, makes it possible to coat also untercuts and passages of the implant surface 3 in an easy and reliable manner. Moreover, the liquid can efficiently dissipate heat generated during iontophoresis.

Outside the coating space 40, the electrodes E1 and E2 are connected to a voltage source 30. Thus, it is possible, by applying an accelerating voltage or a potential difference by means of the voltage source 30, to generate an electric field E between the electrodes E1 and E2, using which the particles T are accelerated. In FIG. 1, this process is illustrated by arrows next to the particles T.

The accelerating voltage for carrying out the reverse iontophoresis is between 10 and 500 V. Preferably, voltages below 200 V are used, and more preferably, voltages below 100 V are used.

In the electric field generated, the charged particles T move from the electrode E1 to the electrode E2, with the particles depositing on the implant I disposed in between and thus being applied to the surface 3 of the implant I. The moving direction of the charged particles T relative to the electric field E depends on whether the particles are charged positively or negatively, which is why the electrodes E1, E2 have to be arranged accordingly in order to apply the coating to the implant surface 3.

It is possible to use plural or differently configured pairs of electrodes. Thus, the field strength of the electromagnetic field E can be influenced. This impact leads in particular to a different acceleration of the particles T. In other words, the acceleration of a charged particle T depends on between which pair of electrodes this particle T is located. In this way, the adhesion and/or the thickness of the coating can be influenced as desired.

The voltage generated by the voltage source 30 can be provided constantly or over a variable period of time. For example, if charged particles T are to penetrate into an implant surface 3 of a porous implant region 10, it can be advantageous to apply a high voltage at first, which can be reduced after the porous cavities have been coated.

Likewise, it can be advantageous to gradually increase the voltage in order to achieve a better adhesion of the coating onto the implant surface 3 of the implant I.

Moreover, the duration of application can be adjusted to the desired coating result. In this way, it is possible to control a penetrating depth and/or a coating thickness with a coating duration or application of the accelerating voltage for a period of 1 minute to 120 minutes.

For example, the implant I shown in FIG. 1 comprises a porous surface region 10, the porosity of which extends into the material 2 of the implant I, preferably up to 1000 μm, more preferably up to 500 μm, and most preferably up to 200 μm.

The porosity of such surfaces is preferably 20 to 60%, and the pore diameter is usually 35 to 800 μm, preferably 50 to 400 μm, more preferably 150 to 200 μm.

By specifically controlling the method parameters of the method according to the invention, it is possible to specifically modify the surface of predetermined regions of the implant surface 3 using iontophoresis.

The implant I to be coated is located in the coating space 40, and it is arranged between the electrodes E1 and E2 disposed opposite each other. As shown in FIGS. 1 and 2, it is possible to divide the coating space 40 into a first subregion 41 and a second subregion 42. In the embodiment shown in FIG. 1, the subregions 41, 42 are separated by a partition wall 44 which is impermeable at least for the charged particles T. In this way, it can be ensured that basically only those portions of the implant I that are to be coated are coated using the method according to the invention. For example, in FIG. 1 the charged particles are located in the subregion 41 facing the implant surface 3.

In this context, it is also possible that part of the partition wall 44 extends onto the implant surface 3 and acts as a mask there, using which it is possible to specifically coat predetermined regions of the implant surface 3. This is advantageous in particular when an electrically conductive surface of the implant as described above is to serve merely as an accelerating electrode for iontophoresis, but electrophoresis is to be prevented.

The particles T are charged particles so that upon application of a voltage at the voltage source 30 these particles are detected by the electric field caused thereby and are accelerated in the direction of the implant surface 3 of the implant I.

In this respect, it is possible that the active ingredient itself forms the charged particle T. Moreover, alternatively or additionally, the active ingredient can be coupled to a charged particle T, for example an ion, so as to allow the method according to the invention to be carried out also with active ingredients which are not charged or which only have a low charge. If an active ingredient only has a low charge, the coating process can thus be accelerated by means of a connection to a particle T having a higher charge.

Of course, it is possible to use also active ingredient combinations for implant coating.

Furthermore, the charged particles T can also be provided for additional tasks during the coating process. For example, they can improve the adhesion of the coating to the implant surface 3.

Likewise, it is possible to implement coating thicknesses by appropriately selecting the charged particles T, in which charged particles T are no longer directly in contact with the implant surface 3 of the implant I. More specifically, the charged particles T form a coating on charged particles T already applied.

It is also conceivable that the charged particles T comprise a hypoallergenic substance as the active ingredient, which, by application to the surface 3 of the implant I, prevents an allergic reaction from being caused by the material 2 of the implant I after implantation in a body.

In a further embodiment of the invention, the charged particles comprise apatite as the active ingredient, for example calcium phosphate or hydroxylapatite, in order to promote the accumulation of osteoprogenitor cells such as osteoblasts or osteocytes. After the implantation and dissolution of apatite in the environment, apatite crystals settle again on the implant surface together with environment proteins by ion exchange.

The implant surface 3 can comprise metal. In such a case, the implant surface can be used as an electrode. Thus, the advantageous aspects of iontophoresis and electrophoresis can be combined.

As shown in FIG. 2, an electrically conductive implant material 15, for example metal or a metal alloy, can be provided below a non-conductive implant surface 13 comprising, for example, a polymer and/or a ceramic. In such an embodiment, at least part of the conductive implant material 15 can be used as the electrode E1 or E2.

Such a way of carrying out the method enables a particularly specific iontophoresis. Depending on whether and to what extent it is possible for the charged particles to penetrate into the implant surface 3, for example in the surface region 110, the coating by iontophoresis can be combined with a partial coating using electrophoresis. The penetrating depth can be predetermined, for example, by the depth of the porous surface region 110 as described above.

For example, it is possible to provide a part of the surface 3, 13 of the implant with an electrode E2 located below the implant surface 3, whereas a different portion of the implant surface is configured with an electrode E2 located directly on the implant surface 3.

1-12. (canceled)

13. A method for coating an implant (I), in particular a prosthesis, by applying charged particles (T) to at least one portion (10, 20; 110, 120) of the implant surface (3, 13) using iontophoresis, wherein the charged particles are accelerated in an electromagnetic field (E) and impinge on the surface (3, 13) of the implant so as to form a coating on at least part of the implant, and wherein the charged particles comprise at least one active ingredient.

14. The method according to claim 13, in which the active ingredient comprises a medicament, in particular an antibiotic.

15. The method according to claim 13, in which the active ingredient comprises a cytokine.

16. The method according to claim 14, in which the active ingredient comprises a cytokine.

17. The method according to claim 13, in which the active ingredient comprises apatite, preferably calcium phosphate and/or hydroxylapatite.

18. The method according to claim 16, in which the active ingredient comprises apatite, preferably calcium phosphate and/or hydroxylapatite.

19. The method according to claim 13, in which at least part of the implant surface is configured to be electrically conductive.

20. The method according to claim 13, in which at least one portion of the implant surface is formed of metal and/or polymer, in particular polyethylene, and or ceramic.

21. The method according to claim 13, in which the accelerating voltage for accelerating the charged particles is at least 10 V and preferably maximally 500 V.

22. The method according to claim 19, in which the accelerating voltage for accelerating the charged particles is at least 10 V and preferably maximally 500 V.

23. The method according to claim 13, in which the accelerating voltage is applied for a period of 1 minute to 120 minutes.

24. The method according to claim 22, in which the accelerating voltage is applied for a period of 1 minute to 120 minutes.

25. The method according to claim 13, in which the strength of the electromagnetic field is changed during application of the charged particles.

26. The method according to claim 24, in which the strength of the electromagnetic field is changed during application of the charged particles.

27. The method according to claim 13, in which the surface (3, 13) of the implant (I) is coated (112) and/or modified before iontophoresis is carried out.

28. The method according to claim 13, in which the implant is provided with a rough and/or porous surface.

29. The method according to claim 27, in which the implant is provided with a rough and/or porous surface.

30. The method according to claim 13, in which the implant is provided with a first layer and a second layer disposed thereabove, the first layer of which has a small pore size for taking up and releasing an antibiotic and the second layer disposed thereabove has a larger pore size compared to the first layer for binding osteoprogenitor cells.

31. The method according to claim 28, in which the implant is provided with a first layer and a second layer disposed thereabove, the first layer of which has a small pore size for taking up and releasing an antibiotic and the second layer disposed thereabove has a larger pore size compared to the first layer for binding osteoprogenitor cells.

32. The method according to claim 29, in which the implant is provided with a first layer and a second layer disposed thereabove, the first layer of which has a small pore size for taking up and releasing an antibiotic and the second layer disposed thereabove has a larger pore size compared to the first layer for binding osteoprogenitor cells.
